(19) United States
(12) Patent Application Publication
(10) Pub. No.: US 2016/0302722 A1
(43) Pub. Date: Oct. 20, 2016

(54) THERMO DETECTOR TO MEASURE MASS

(71) Applicant: REHAM ASHKANANI, AL-BEDAE (KW)

(72) Inventor: REHAM ASHKANANI, AL-BEDAE (KW)

(21) Appl. No.: 14/686,680

(22) Filed: Apr. 14, 2015

(51) Int. Cl. A61B 5/00; A61B 5/107; A61B 5/01

(52) U.S. Cl. CPC A61B 5/4872; A61B 5/0075; A61B 5/015; A61B 5/4519; A61B 5/7278; A61B 5/1079; A61B 5/7475; A61B 5/0082; A61B 2576/00; A61B 2560/0406; A61B 2562/227; A61B 2560/0431; A61B 2562/0233

(57) ABSTRACT

The thermo detector to measure mass includes a housing for holding an infrared sensor for detecting infrared radiation emitted by a patient. The emitted infrared radiation is used to distinguish between fat and muscle of the patient in a selected body part based upon variations in temperature represented by the detected infrared radiation. A laser is further disposed in the housing for projecting a scanning laser beam on the selected body part of the patient, and at least one laser sensor receives a reflected laser beam from the selected part of the patient. From the reflected laser beam, a volume of the fat and a volume of the muscle of the selected body part based on the reflected laser beam are determined. A mass of the fat and a mass of the muscle in the selected body part of the patient are then determined based on the detected volumes thereof.

### BACKGROUND OF THE INVENTION

1. Field of the Invention

The present invention relates to a system and method for body measurement, and particularly to a thermo detector to measure muscle mass and body fat.

2. Description of the Related Art

The connection between muscle gain and weight loss is probably one of the most misunderstood aspects of health and fitness. Adding muscle to a person's body helps the person to lower their body fat percentage in a variety of ways. However, these effects are often indirect and can be confusing. There is also misunderstanding regarding whether a person is physically fit based on body weight provided by a scale. These scales provide a measure of total weight, but don't determine the lean-to-fat ratio of that weight. Standing on most scales can tell you only if you weigh more than the average person, but not if that weight is fat or muscle. Based only on scale weight, a 250-pound athlete with 8% body fat may be considered “overweight” by a typical weight chart. Such charts are not good indicators of ideal body weight for general health or for athletic performance. There are other methods of assessing a person's body fat percent and lean mass to determine general body health.

A device that provides accurate measurement of muscle mass and body fat would be desirable for monitoring a person's health and fitness. Thus, a thermo detector to measure mass solving the aforementioned problems is desired.

### SUMMARY OF THE INVENTION

The thermo detector to measure mass includes a housing for holding an infrared sensor, such as a thermographic camera, infrared camera, thermal imaging camera or the like, for detecting infrared radiation emitted by a patient. The emitted infrared radiation is used to distinguish between fat and muscle of the patient in a selected body part based upon variations in temperature represented by the detected infrared radiation. A laser is further disposed in the housing for projecting a scanning laser beam on the selected body part of the patient, and at least one laser sensor, also disposed in the housing, receives a reflected laser beam from the selected part of the patient. From the reflected laser beam, a volume of the fat and a volume of the muscle of the selected body part based on the reflected laser beam are determined. A mass of the fat and a mass of the muscle in the selected body part of the patient may then be determined based on the detected volumes thereof.

The thermographic camera detects infrared radiation that are emitted by the human body. The laser sensor detects the reflected laser radiation from the laser spot projected onto the patient. Under certain controlled conditions, the infrared radiation will depict the muscle mass and body fat by taking the temperature of a specific body part, for example, the biceps. The temperature of the specified muscle will be analyzed and converted to two parts, i.e., the muscle and fat percentages of the individual's body. The muscle and fat percentages will be determined by the temperature readings, where the higher temperatures will read as the muscle and the lower temperatures will read as the fat for the specified body part. The laser radiation will take the volume of the muscle and/or fat in three dimensions (3D), e.g., depth, width, and length, in cross sections. The processor in the thermo detector processes the data obtained by the various sensors and cameras. The processor is able to calculate the length, width, and depth of the muscle or specified body part by using a computer program or algorithm that splits the data into a number of cubes, such as cubes the size of 1 mm³. The program or algorithm can determine the muscle mass and body fat based on calculating the number of cubes. The calculated data processed by the processor will be produced, such as on a digital display or to a photo printer. The data and images can be also be sent over wireless or hardwire connections to other users or systems. The thermo detector may be disassembled into component parts and adapted for transport.

These and other features of the present invention will become readily apparent upon further review of the following specification and drawings.

### BRIEF DESCRIPTION OF THE DRAWINGS

FIGS. 1A and 1B are environmental, perspective views of a thermo detector to measure mass according to the present invention, shown in use to detect the mass in a particular body region.

FIGS. 1C, 1D, and lE are schematic diagrams illustrating sectioning tissue to determine the detected mass in the specified body region using a thermo detector to measure mass according to the present invention.

FIG. 2A is a front view of the thermo detector of FIG. 1 A.

FIG. 213 is a detail view of area 2B of FIG. 2A.

FIG. 3A is a partial perspective view of a thermo detector to measure mass according to the present invention, showing a remote control for operating the thermo detector.

FIG. 3B is another partial perspective view of the thermo detector of FIG. 3A, showing the rear of the housing.

FIG. 3C is a perspective view of a remote control for operating the thermo detector to measure mass according to the present invention.

FIG. 4A is a perspective view of a thermo detector according to the present invention, shown disassembled for storage and transport.

FIG. 4B is a perspective view of a portable carrying case for transporting a thermo detector to measure mass according to the present invention.

FIG. 5A is a perspective view of the portable carrying case of FIG. 4B, shown with the front cover open to reveal the disassembled thermo detector of FIG. 4A packed inside of the case.

FIG. 5B is a perspective view of the portable carrying case of FIG. 5A with the front cover closed.

FIG. 6 is a flowchart showing the steps of a method for operating a thermo detector to measure mass according to the present invention

FIG. 7 is a block diagram of a control unit for a thermo detector to measure mass according to the present invention.

Similar reference characters denote corresponding features consistently throughout the attached drawings.

### DETAILED DESCRIPTION OF THE PREFERRED EMBODIMENTS

The thermo detector to measure mass may be used to accurately measure a person's muscle mass and body fat. The thermo detector includes thermographic cameras, such as infrared sensors or laser sensors, a laser for generating a laser spot, a number of power sources, a printing device, an external display connection, a computer processor, a computer memory, and remote control. The remote control is used to operate the system and the various components used to measure mass.

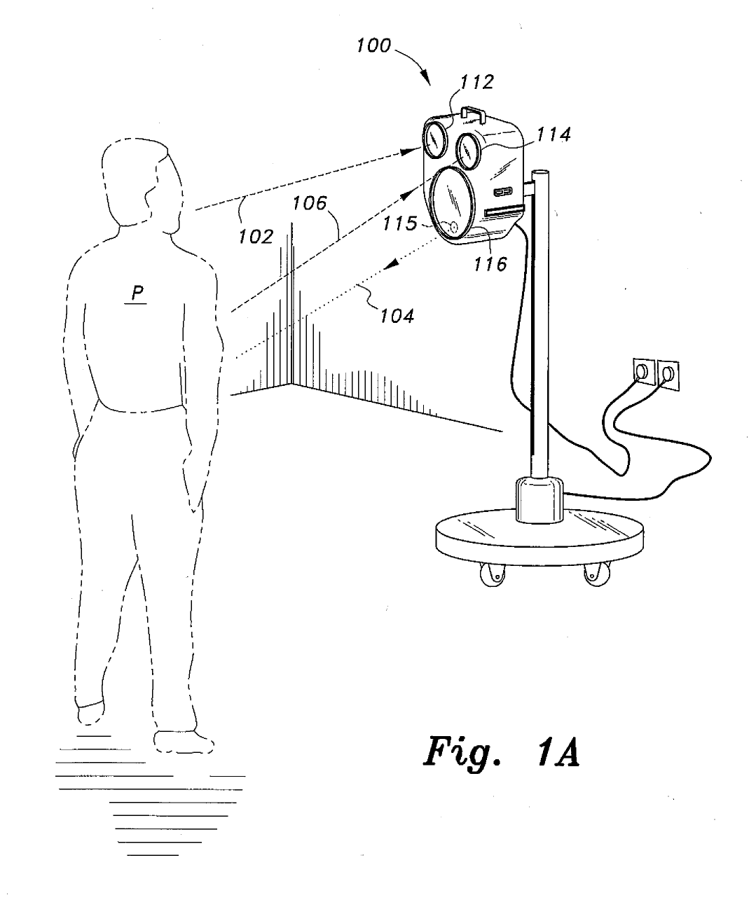

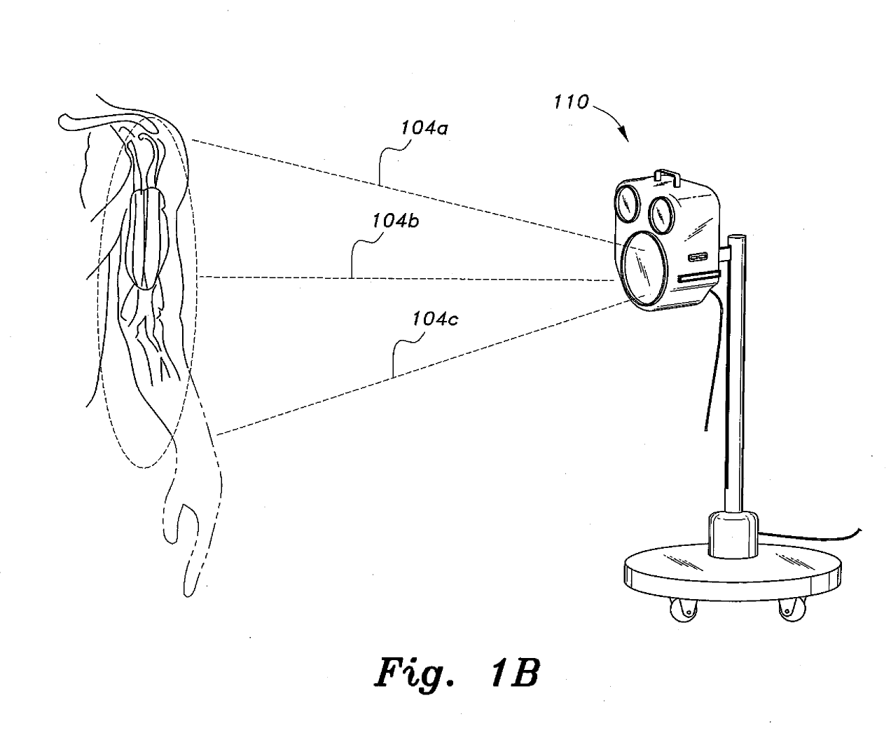

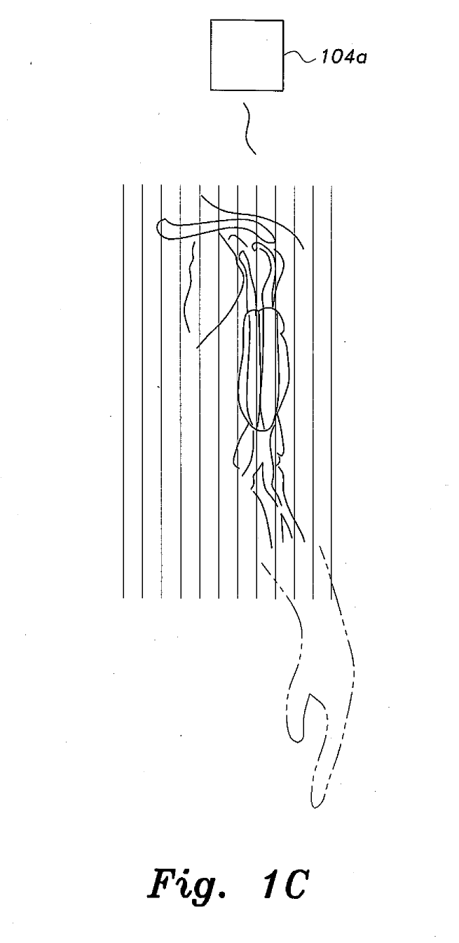

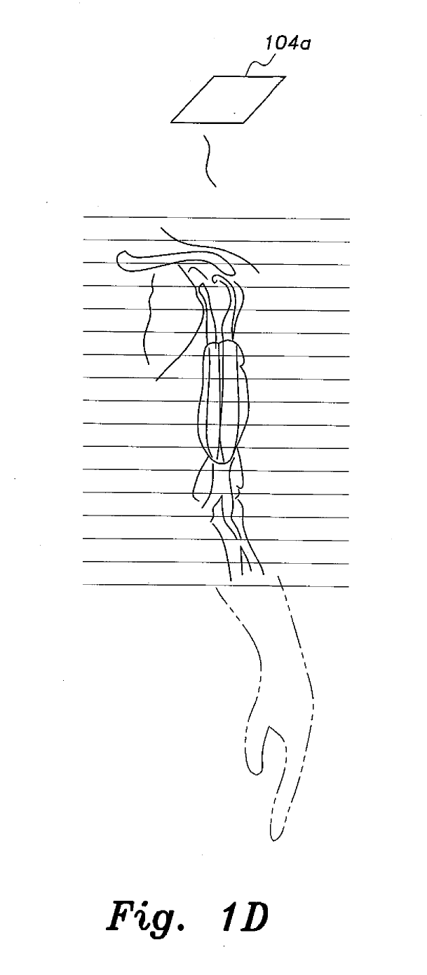

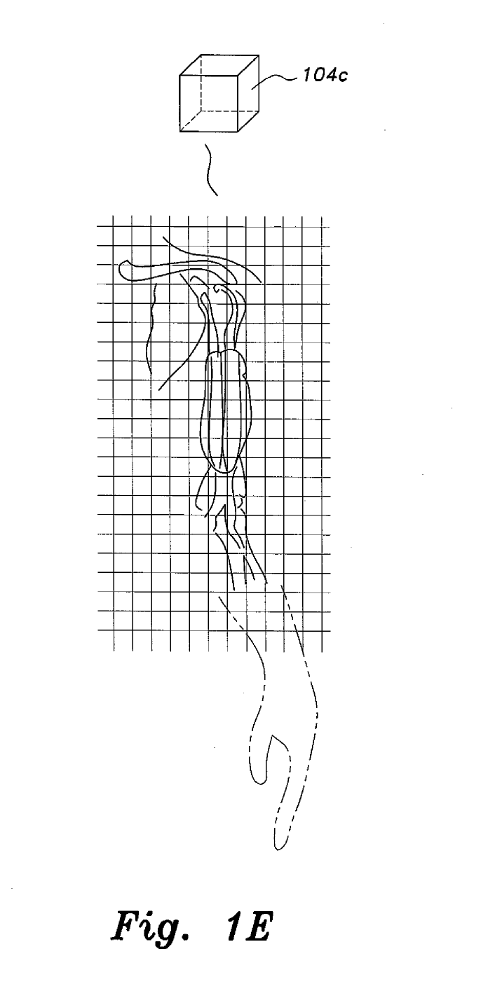

FIGS. 1A and 1B show the thermo detector 100 to measure mass that uses both infrared radiation and laser beams to detect the mass in a particular body region. The thermo detector 100 of FIG. 1A receives infrared radiation 102, and further includes a laser 115 for generating a laser beam 104, which is projected to the patient P for scanning the patient P. The laser beam 104 emitted by the laser 115 is reflected back as reflected beam 106 to a laser sensor 114 to determine the volume of the muscle and body fat of patient P. FIG. 1B illustrates a detailed view of the laser beams 104 in a comprehensive display where multiple laser beams 104a, 104b, and 104c are used to cover a specified body region.

FIGS. 1C-1E illustrate a detailed view of the detected mass in the specified body region in sections to calculate the volume of muscle and body fat. FIG. 1C illustrates the beam 104a that will scan the specified body region in a vertical method. FIG. 1D illustrates the beam 104b that will scan the specified body region in a horizontal method. FIG. 1E illustrates the beam 104a that will scan the specified body region in a cross-sectioned method for calculating the volume of muscle and body fat.

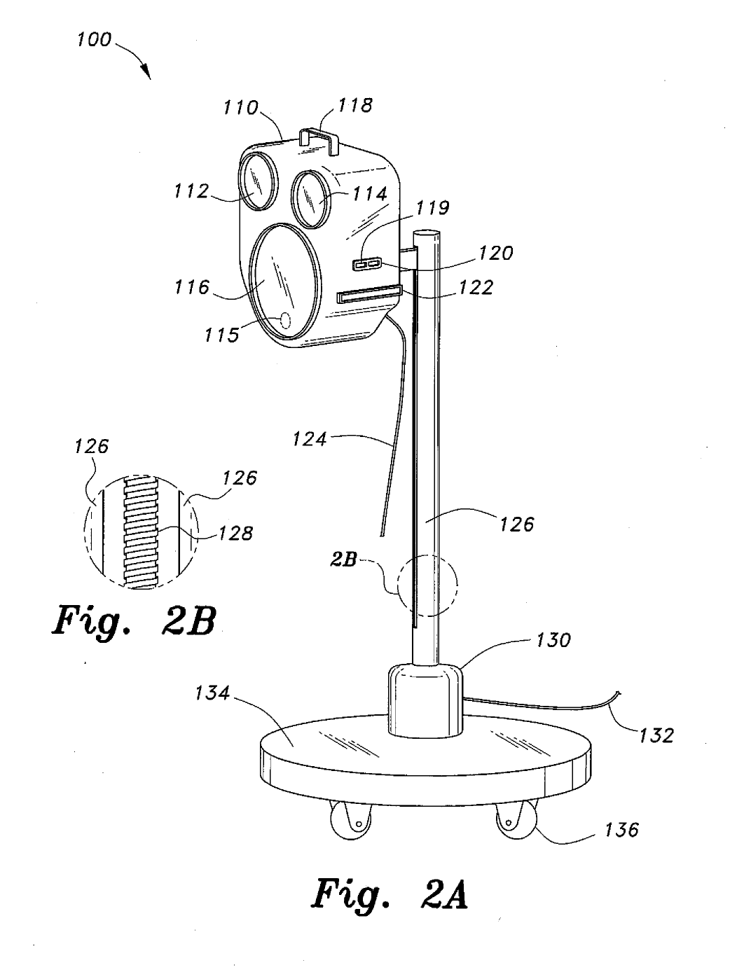

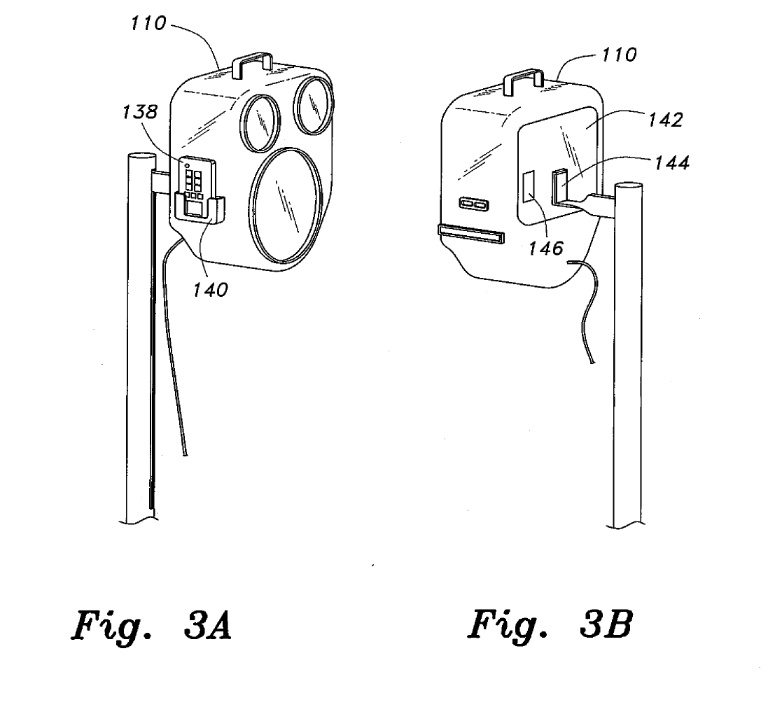

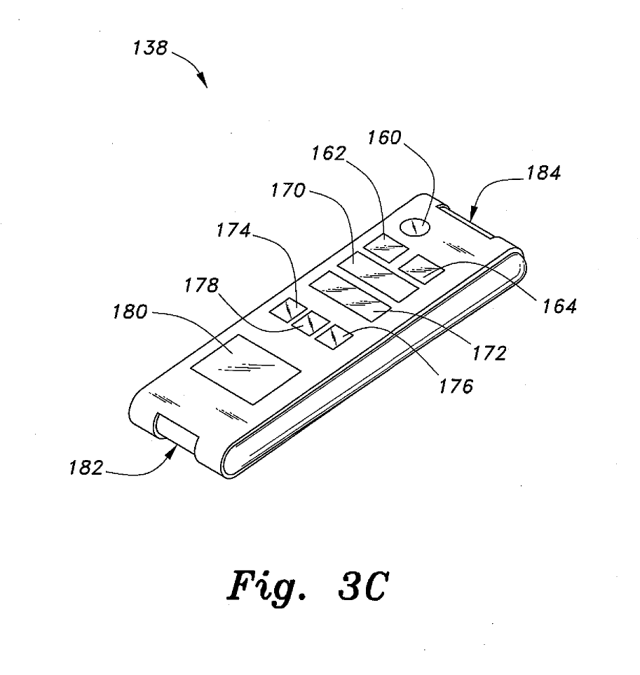

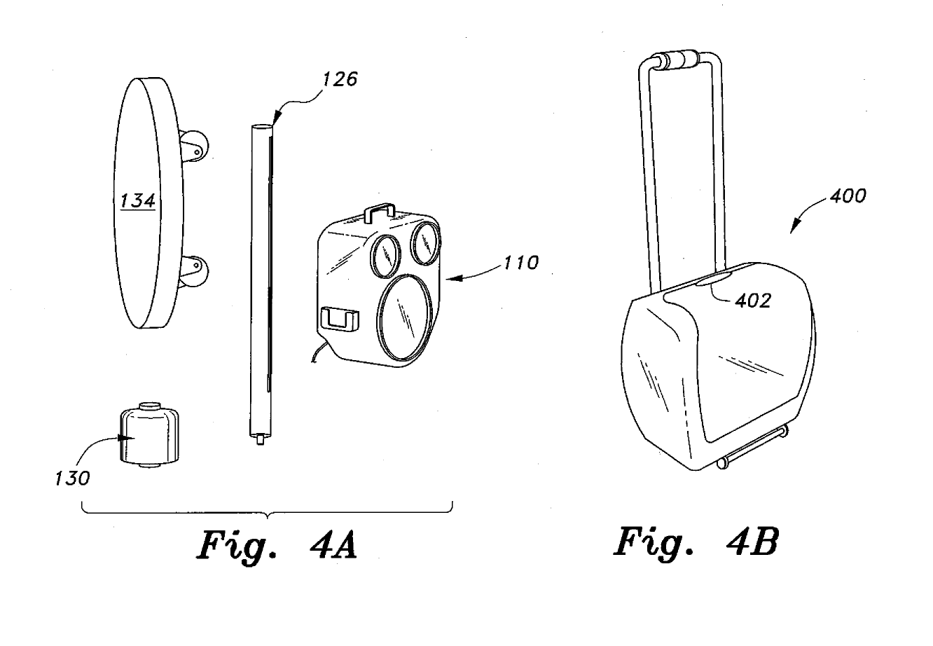

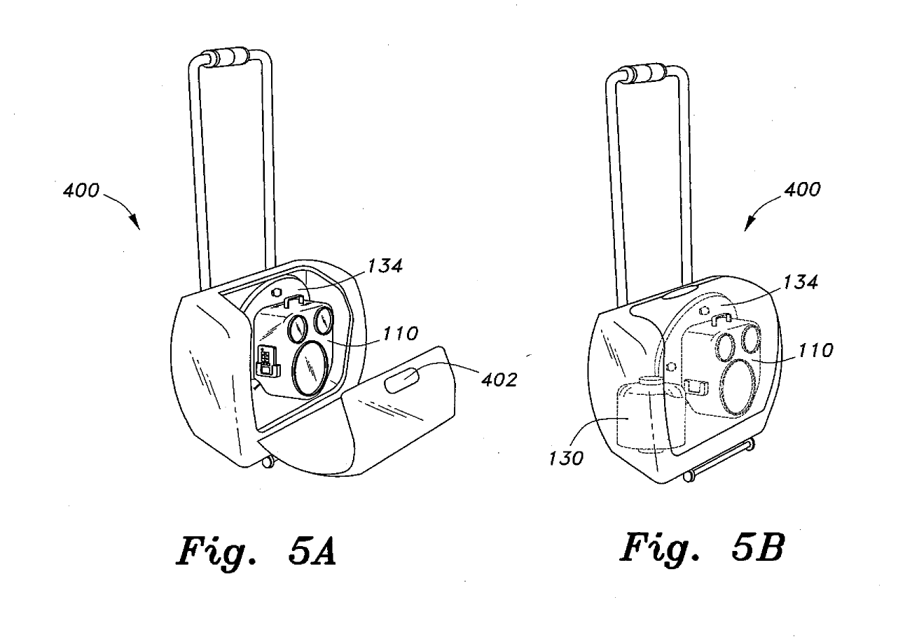

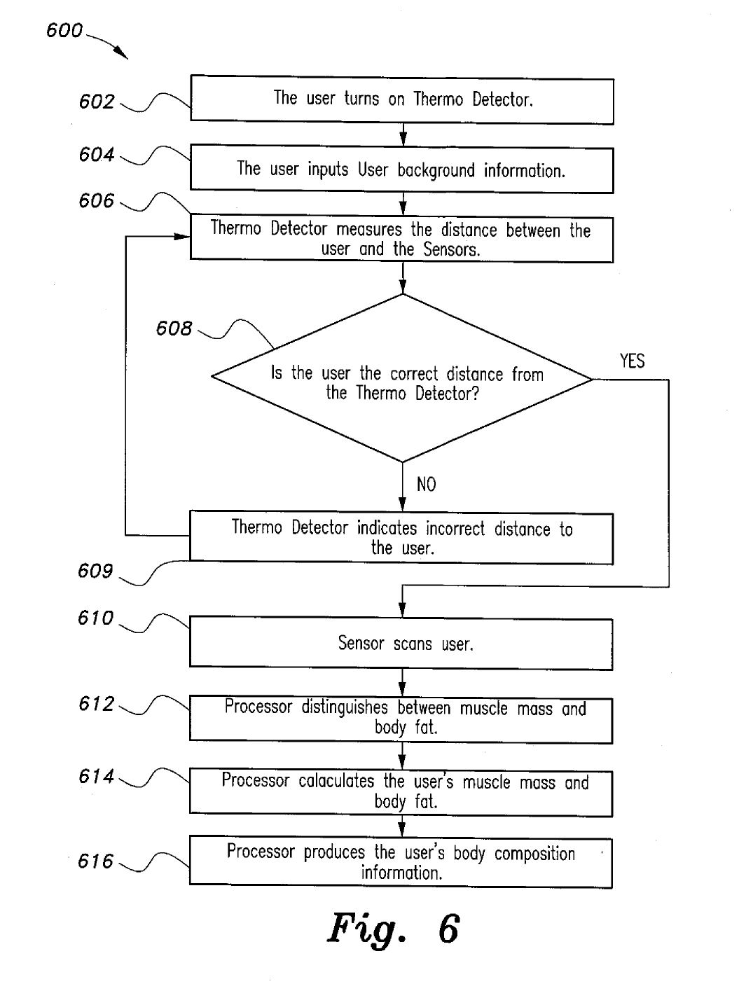

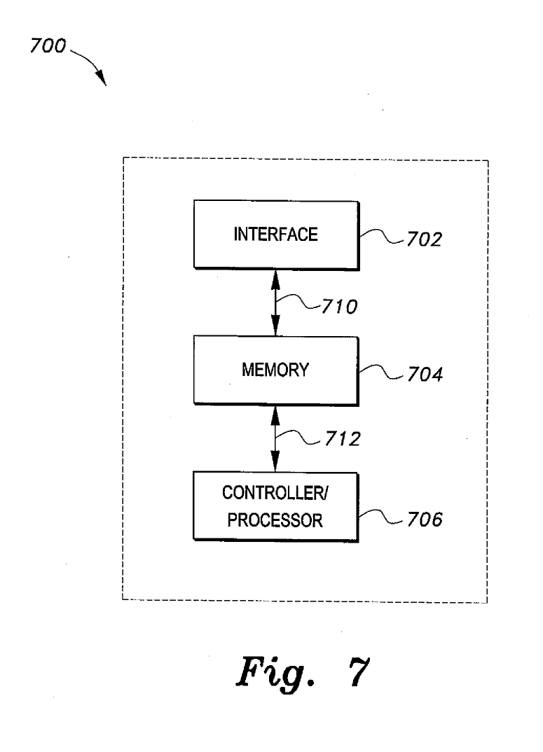

FIG. 2A illustrates a front view of the thermo detector 100 for detecting a person's body composition. FIG. 2A shows the body casing (housing) 110 of the thermo detector 100, which contains the laser 115, along with a distance sensor 112, a laser sensor 114, an infrared sensor 116, such as a thermographic camera, infrared camera, thermal imaging camera or the like, a USB port 119, a handle 118, an external screen port 120, a photo printer 122, and a power supply cord 124. It should be understood that any suitable type of distance sensor, laser sensor and infrared sensor may be utilized. FIG. 2A also illustrates a support pole 126 to support the body casing of the thermo detector 110. The body casing 110 of the thermo detector 100 and the support pole 126 can be made of any material and is not limited to plastic or metal.

FIGS. 2A and 2B illustrate an elevating spring 128 inside of the support pole 126, an adapter 130 for the support pole 126, a power supply cord 132, a base stand 134 for the thermo detector, and a plurality of wheels 136. The power supply cords 124 and 132 are ideally used with alternating current (AC) at 240 volts, but the device may also be made for operation on a 120 V AC power main. The infrared and laser sensors are powered by a separate power supply; e.g. the power supply cord 124, than the power source of the thermo detector, such as the power supply cord 132.

FIG. 3A illustrates a partial perspective view of a thermo detector 100 to measure mass, including a remote control 138 for operating the thermo detector 100. FIG. 3A illustrates the body casing (housing) 110 of the thermo detector 100, remote control 138, and a remote control holder 140.

FIG. 3B illustrates another partial perspective view of a thermo detector 100 to measure mass. FIG. 3B illustrates the body casing (housing) 110 of the thermo detector 100, a battery cover 142, a battery cover release 146, and a mounting slot 144.

FIG. 3C illustrates a perspective view of the remote control 138 for operating the thermo detector 100. FIG. 3C illustrates the remote control 138 and various buttons used to operate the thermo detector 100. The various buttons on the remote control 138 include a power button 160 to turn the thermo detector 100 on or off, a down button 162, an up button 164, a button 170 for clarifying the infrared image, a button 172 for turning the laser on or off, laser adjustment buttons 174, 176, and 178, a shutter control button 180, a battery cover 182, and an LED 184.

FIG. 4A illustrates the thermo detector 100 of FIG. 2 disassembled into several components. The body casing 110 of the thermo detector 100 is no longer affixed to the support pole 126. The adapter 130 for the support pole 126 is no longer affixed to the base stand 134 for the thermo detector 100.

FIG. 4B illustrates a traveling case 400 for the thermo detector 100. The traveling case 400 can be made of any suitable material, such as cloth, fabric, plastic, or a combination thereof and is not limited in this regard. The traveling case 400 has a latch 402 to open the traveling case 400 for storing the disassembled components of the thermo detector 100. The placement of the latch 402 can be in any location on the traveling case 400 and is not limited in this regard. The latch 402 can also be made of any suitable material, such as plastic or metal, but is not limited in this regard. Furthermore, access to the interior of the traveling case 400 can be accomplished by other fastening means, such as a zipper, clasp, or button, and is not limited in this regard.

FIG. 5A illustrates the traveling case 400 with the latch 402 opened to contain the disassembled components of the thermo detector 100, such as the thermo detector body casing 110 and the base stand 134, placed inside of the traveling case 400. FIG. 5B illustrates disassembled components of the thermo detector 100 inside of the traveling case 400 with the latch 402 closed.

Referring now to FIG. 6, a flowchart of a method for operating the thermo detector 100 is shown. At step 602, the user turns on the thermo detector 100. Prior to operating the thermo detector 100, it is optimal for the subject to be nude for approximately twenty minutes to allow the body to equilibrate to room temperature without affect from clothing, water, perspiration, lotion, or liquid. Additionally, the subject should not engage in any workout or physical exertion prior to the examination, as physical effort can change the temperature, length, mass and weight of muscle. This physical effort can distort the readings of the thermo detector 100. It is also ideal to conduct the examination with the thermo detector in a room with dimmed lighting and a temperature approximately 20° C.-25° C. Furthermore, the room should not contain any objects or devices that generate heat that could interfere with the camera receptor in the thermo detector 100.

Continuing at step 604, the user inputs the subject's background information, such as gender, age, height, and weight. At step 606, the thermo detector detects the distance between the person being measured and the thermographic camera inside of the thermo detector. The distance between the thermo detector and the subject should ideally be thirty centimeters to avoid side effects of infrared radiation, yet still also provide quality images. At step 608, a processor, such as the controller/processor 706 of FIG. 7, checks whether the subject is the correct distance away from the thermo detector, e.g. 30 centimeters. If the subject is not the correct distance away, the thermo detector can notify the user of the incorrect distance at step 609, and the process returns to step 606. If the subject is the appropriate distance at step 608, the process proceeds and the sensors in the thermo detector scan the subject at step 610.

Continuing at step 612, the processor 706 in the thermo detector 100 distinguishes the muscle mass from the body fat. The differentiating between the muscle and fat can be done by a number of methods, but generally requires a thermographic camera or the like that is able to distinguish different wavelengths of infrared radiation, such as infrared or laser radiation. In addition to distinguishing the muscle and fat, the sensors can simultaneously and independently measure a person's muscle mass and body fat. The thermodetector camera, which is preferably a 640×480 pixel camera, can detect the radiation, such as infrared radiation, to a wavelength of 3-5 micrometers to measure muscle temperature.

The thermographic camera in the thermo detector 100 has one setting button that is used to modify and obtain the person's body composition. The button determines the body heat temperature of a specific area, such as a muscle. Under controlled conditions, the laser radiation obtained by the various zoom buttons will be used to take the volume of muscle and/or fat in three dimension (3D), e.g., depth, width, and length, in cross sections. The infrared and laser radiations will also depict the muscle mass and body fat by taking the temperature of a specific muscle, for example, the biceps. The infrared and laser radiations will be used to display images of the scanned area, such as a 3D image and thermographic image.

Once the user's muscle mass and body fat has been differentiated, the processor 706 in the thermo detector 100 calculates the data of the user's muscle mass and body fat at step 614. The processor 706 in the thermo detector 100 is the component in the thermo detector 100 that processes and calculates the data obtained by the various sensors and cameras. The processor 706 displays the image and uses a color pixel counter program to count the pixels. The processor 706 is able calculate the volume of the muscle and the body composition. The processor 706 determines the muscle mass and body fat based upon these calculations. Finally, the processor 706 produces the user's body composition at step 616. The processor 706 can send the calculations of the person's muscle mass and body fat percentage to be displayed on to a digital display, such as through the external screen port 120 or to the photo printer 122.

The type of thermographic camera used in the thermo detector is preferably a cooled thermo graphic camera, but can be any type of thermographic camera adapted for use in thermal imaging and infrared radiation use and can include various types of infrared image detectors, such as cooled and uncooled detector. The optimal frequency to use the thermo detector 100 to detect muscle mass and body fat is 60 Hz, the optimal range is 3-5 micrometers, but is not limited to this, and can include other ranges and frequencies, and the image resolution is preferably 640×480 pixels. Once the thermo detector 100 has obtained the measurements, the measurements are calculated to determine the exact percentage of muscle to fat ratio in the scanned region. The muscle mass and body fat can be calculated through an algorithm or program that is adapted for use to calculate the muscle mass and body fat based on the information obtained by the sensors, thermographic camera, and the information entered by the user, such as a color pixel counter program.

In FIG. 7, the generalized system 700 includes an interface 702, a memory 704, and a controller/processor 706, for example. Information, such as body mass information from the sensors, can be acquired by the interface 702 through a number of sensors and stored at 710 in the memory 704, such as a computer readable memory, which can be any suitable type of non-transitory computer readable and programmable memory.

Examples of computer readable media as can be used or included in the memory 704 can include a non-transitory computer readable storage memory, a magnetic recording apparatus, an optical disk, a magneto-optical disk, and/or a semiconductor memory (for example, RAM, ROM, etc.). Examples of magnetic recording apparatus that may be used in addition to memory 704, or in place of the memory 704, include a hard disk device (HDD), a flexible disk (FD), and a magnetic tape (MT). Examples of the optical disk include a DVD (Digital Versatile Disc), a DVD-RAM, a CD-ROM (Compact Disc-Read Only Memory), and a CD-R (Recordable)/RW.

For example, information or data can be transmitted from or received by the interface 702, such as received sensor data and information as to a measurement of muscle mass and body fat. Such information or data can be organized in the memory 704 and transmitted to or from the memory 704, such as a computer readable memory, at 712 to the controller/ processor 706 or at 710 to the interface 702, The interface 702 can include connection to an external digital display or to the photo printer. The data can also be transferred or obtained by USB connection or any suitable form that allows data transfer.

Operations are performed by the controller/processor 706, which can be any suitable type of computer implemented device, such as a computer processor, as discussed. Also, the resulting information, resulting data or resulting determination made by the controller/processor 706 from the information or data processed by the controller/processor 706 can be stored in the memory 704 and can be transmitted through the interface 702, such through the external screen port 120 to a digital display or to the photo printer 122. The photo printer 122 or the digital display can show the exact muscle mass measurements, including the volume of the muscle, for example, calculated by the color pixel counter program. The photo printer 122 or the digital display can also show the exact body fat percentage of the body area scanned.

The information and operations that are transmitted throughout the various embodiments of a thermo detector 100 to measure mass or methods for operating a thermo detector can be in the form of electronic data, wireless signals, or a variation thereof The information and operations that are transmitted throughout the various embodiments can be sent wirelessly, optically, or by any of various types or arrangements of hard-wire connections, or combinations thereof, among the various system components, for example.

It is to be understood that the present invention is not limited to the embodiments described above, but encompasses any and all embodiments within the scope of the following claims.

1. A thermo detector to measure mass in a selected body part of a patient, comprising:
a housing;
a distance sensor disposed in the housing, wherein the distance sensor is configured to determine the correct distance of the patient from the thermo detector in order to initiate sensor scannings of the selected body part of the patient;
an infrared sensor disposed in the housing for detecting infrared radiation emitted by a patient;
means for distinguishing between fat and muscle of the patient in the selected body part based upon variations in temperature represented by the detected infrared radiation, wherein the higher temperatures represent muscle and the lower temperatures represent fat, the means for distinguishing between fat and muscle further includes a non-transitory computer readable medium with a computer program product embodied thereon, the medium having stored thereon a set of instructions for determining the percentages of fat and muscle and displaying the results, the set of instructions including at least a first sequence of instructions which, when executed by the processor, causes the processor to receive the temperatures of the selected body part;
a laser disposed in the housing for projecting a scanning laser beam on the selected body part of the patient;
at least one laser sensor disposed in the housing for receiving a reflected laser beam from the selected part of the patient;
means for determining a volume of the fat and a volume of the muscle of the selected body part based on the reflected laser beam, wherein the means for determining the volume of fat and muscle further includes a non-transitory computer readable medium with a computer program product embodied thereon, the medium having stored-thereon a set of instructions for determining the volume of fat and muscle and displaying the results as a 3D representation, the set of instructions including at least a first sequence of instructions which, when executed by the processor, causes the processor to receive the 3D representations and to display the results of the selected body part; and
means for determining a mass of the fat and a mass of the muscle in the selected body part of the patient based on the detected volumes thereof, wherein the means for determining the mass of the fat and muscle further includes a non-transitory computer readable medium with a computer program product embodied thereon, the medium having stored-thereon a set of instructions for determining the mass of fat and muscle and displaying the results, the set of instructions including at least a first sequence of instructions which, when executed by the processor, causes the processor to receive the results developed by the infrared and laser sensors and to display the results thereof.

2-10. (canceled)

11. A method for measuring muscle mass and body fat in a portion of a human body, comprising the steps of:
obtaining a thermographic image of a portion of a human body with a thermographic camera;
identifying muscles and body fat in the thermographic image;
scanning the identified muscles with laser radiation to determine a volume of the muscles;
computing the mass of the muscles from the volume of the muscles;
computing the volume of body fat from the thermographic image; and
computing the percentage of body fat from the volume of the muscles and the volume of the body fat.
